(19) United States
(12) Patent Application Publication
(10) Pub. No.: US 2021/0100472 A1
(43) Pub. Date: Apr. 8, 2021

(54) INTRACARDIAC CATHETER DEVICE AND METHODS OF USE THEREOF

(71) Applicant: ASAHI INTECC Co., LTD, Seto-shi (JP)

(72) Inventors: Fumiyoshi Oshima, Nagoya (JP); Wayne Ogata, San Ramon, CA (US); Rei Uemura, San Jose, CA (US)

(21) Appl. No.: 17/064,928

(22) Filed: Oct. 7, 2020

Related U.S. Application Data
(60) Provisional application No. 62/912,039, filed on Oct. 7, 2019.

(51) Int. Cl. A61B 5/05; A61B 5/00; A61B 5/06; G01R 33/10

(52) U.S. Cl. CPC A61B 5/05; A61B 5/743; A61B 5/6851; A61B 2562/028; A61B 5/062; G01R 33/10; A61B 5/6852

(57) ABSTRACT

An apparatus includes a longitudinal member having a proximal end and a distal end. The longitudinal member is configured to be located near a tissue region in a body of a patient. A measuring device is configured and sized to be located proximal to the distal end of the longitudinal member. The measuring device includes a magnetic sensor configured to measure biomagnetism and output magnetic flux data. A signal processing device is coupled to the magnetic sensor and configured to convert the output magnetic flux data to a digital representation of the output magnetic flux data. A method of measuring electrical activity using the apparatus is also disclosed.

This application claims the benefit of U.S. Provisional Patent Application No. 62/912,039 filed Oct. 7, 2019 the entirety of which is incorporated herein by reference.

### FIELD

The present technology relates to an intracardiac catheter device and methods of use thereof and, more specifically to an intracardiac catheter device for mapping cardiac activity using magnetophysiology.

### BACKGROUND

Mapping of cardiac activity may be utilized to treat heart conditions, such as arrhythmia. Various techniques have been employed to provide such cardiac mapping. For example, electrocardiograms (ECGs) utilize electrodes to measure electrical activity of the heart. In a typical ECG procedure, external electrodes are placed on the surface of the patient's body to measure the electrical activity of the heart from a variety of angles.

Alternatively, an electrode attached to the tip of a catheter can be utilized to provide intracardiac measurements by contacting the endocardium. An ECG combining extracardiac and intracardiac heart measurements may also be employed to measure electrical activity of the heart. Using an ECG, the electrical activity of the heart can be mapped to determine the existence of abnormalities, such as an arrhythmia by way of example. However, measurements using electrodes are impacted by the electrical activity of other tissues in the body and generally require that the electrodes are in direct contact with the tissue. Thus, ECG techniques cannot elucidate the fine electrical excitation sequence of the heart to obtain detailed location data for abnormalities that can be utilized for treatment.

In recent years, intracardiac measurements using electrodes attached to catheters have been combined with an extracardiac magnetocardiogram (MCG). These techniques provide for a more accurate determination of the location of the occurrence of an abnormality, such as an arrhythmia, to a level of accuracy that can be put to practical use in treatment. It has been reported that by performing an ECG along with an external MCG measurement simultaneously, the diagnostic success rate can be increased by an average of 50% compared to the method using only the ECG, depending on the type of the condition.

However, employing an MCG relies on magnetphysiology, which involves measuring the magnetic field generated by the ionic currents produced by cardiac activity. However, the magnetic fields at the surface of the body are weak. These signals are typically seven to nine orders of magnitudes lower than the Earth's magnetic field and five orders of magnitude lower than the environmental magnetic noise. Thus, an ultra-sensitive magnetic sensor is required.

Hypersensitive magnetic sensors, such as sensors that employ SQUID (Superconducting Quantum Interference Devices) have been utilized to determine the location of myocardial excitation transfer abnormality in three dimensions. Because these sensors are large, they must be used to measure the magnetic field from outside of the body. Further, measuring these weak magnetic fields externally requires a shielded environment, and the SQUID sensors require nitrogen or helium liquid cooling. Thus, the current systems utilized for MCG are very expensive and complicated, limiting their use.

### SUMMARY

An apparatus includes a longitudinal member having a proximal end and a distal end. The longitudinal member is configured to be located near a tissue region in a body of a patient. A measuring device is configured and sized to be located proximal to the distal end of the longitudinal member. The measuring device includes a magnetic sensor configured to measure biomagnetism and output magnetic flux data. A signal processing device is coupled to the magnetic sensor and configured to convert the output magnetic flux data to a digital representation of the output magnetic flux data.

A method for measuring electrical activity includes receiving, by a computing device, magnetic flux data from a measuring device positioned on a longitudinal member having a proximal end and a distal end, wherein the longitudinal member is configured to be located near a tissue region in a body of a patient and the measuring device is located proximate to the distal end. The magnetic flux data is based on electrical activity near the tissue region. A magnetic flux distribution is generated, by the computing device, for the tissue region based on the magnetic flux data.

This technology provides a number of advantages including providing a very small, ultra-sensitive three-dimensional magnetic sensor that may be employed on a catheter to measure the three-dimensional magnetic flux within a patient's body without necessitating direct contact with the tissue. By way of example, the device may be employed in an intracardiac procedure to measure the magnetic flux distribution in the endocardial membrane. The device advantageously can map changes of the three-dimensional magnetic flux distribution in the endocardial membrane in real-time and display it with spatial contours. Thus, the technology allows for the identification of the source of an arrhythmia. In addition, the position of the catheter is measured by an ultra-small, three-dimensional magnetic sensor that can measure the geomagnetism or biomagnetism to improve the accuracy of the determination of the location of the abnormality.

### BRIEF DESCRIPTION OF THE DRAWINGS

FIG. 1 is an exemplary environment including an exemplary intracardiac mapping system including an intracardiac device coupled to a computing device.

FIG. 2 is an illustration of the exemplary intracardiac catheter located in a patient's heart to measure electrical activity.

FIG. 3 is an illustration of the magnetic sensor device used in the intracardiac catheter.

FIG. 4 is a block diagram of the computing device illustrated in FIG. 1.

FIG. 5 is a flow chart of an exemplary method of mapping cardiac activity using the intracardiac catheter device.

FIG. 6 is an illustration of an exemplary deflectable catheter comprising a basket configuration on the distal end and comprising multiple magnetic sensors of the present technology.

FIG. 7 is an exemplary catheter with a distal end comprising multiple magnetic sensors of the present technology.

FIG. 8 is an exemplary guidewire with a distal end comprising a magnetic sensor of the present technology.

### DETAILED DESCRIPTION

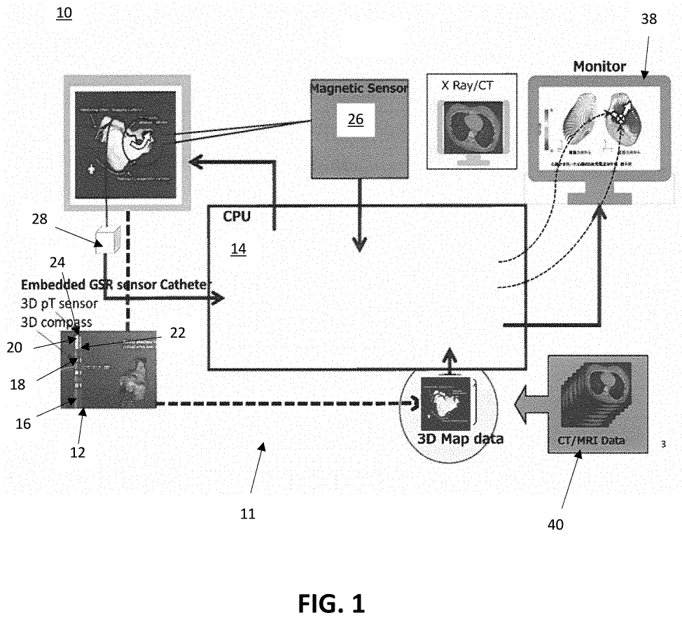

An exemplary environment 10 including an exemplary system 11 for measuring and mapping cardiac activity is illustrated in FIGS. 1-4. The system 11 includes the intracardiac catheter device 12, which includes a longitudinal member 16 having a measurement device 18 and a position sensor 20 disposed thereon, and the computing device 14, although the system 11 could include other types and/or numbers of devices, components, and/or other elements in other configurations, such as imaging devices or server devices. This exemplary technology provides a number of advantages including providing more efficient methods of measuring and mapping cardiac activity for use in the identification and treatment of abnormalities.

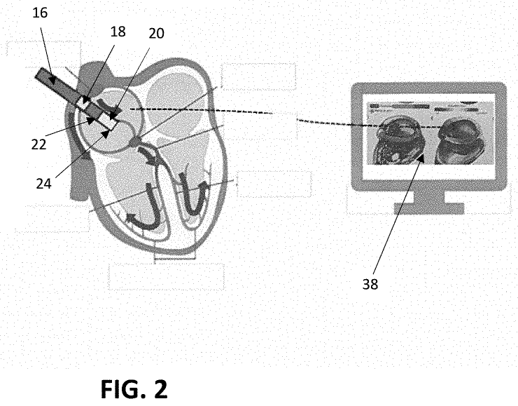

Referring more specifically to FIGS. 1 and 2, the system 11 includes the longitudinal member 16, which extends between a proximal end (not shown) and a distal end 22. The longitudinal member 16 is configured to be advanced into the body of a patient and located near a tissue region. In this example, the longitudinal member 16 is sized and configured for intracardiac placement, although the longitudinal member 16 may be utilized for placement in other tissue regions of the patient, such as other organs, body lumens or cavities, such as various ducts or vessels, or blood vessels by way of example only. The longitudinal member 16 may be placed near the tissue region using various approaches and orientations, such as retrograde and antegrade approaches. In this example, the longitudinal member 16 is a catheter, although other types and/or numbers of longitudinal members that can be inserted into the body, such as by way of example only guidewires, micro catheters, dilating catheters, or probes, may be utilized.

The longitudinal member 16 includes the measurement device 18 located near the distal end 22 of the longitudinal member 16, although the longitudinal member may also include other devices located near the distal end 22, such as a permanent magnet, a positional sensor, additional magnetic sensors, a pressure sensor, a temperature sensor, a contact force sensor, a torque or rotational sensor, or motion sensors including gyroscopes and accelerometers, as described below. In one example, the measurement device 18 is located on a distal tip 24 of the longitudinal member. Incorporating the measurement device 18 in a catheter, by way of example, allows the measurement device 18 to be placed in the heart, for example, to measure a stronger signal near the source, although the measurement device 18 may be used in other applications including for example to measure blood flow in blood vessels or to characterize different tissue types by distinguishing differences in the strength of the magnetic field based on tissue characteristics (biomagnetism).

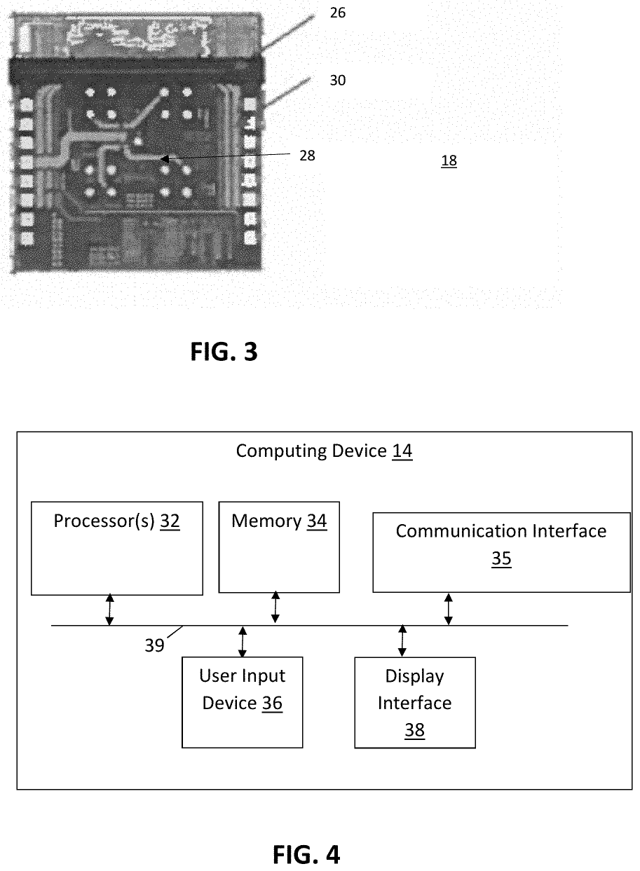

Referring now to FIG. 3, the measurement device 18 is illustrated. In this example, the measurement device 18 includes a magnetic sensor 26 coupled to a signal processing device 28 including an integrated circuit 30 configured to convert analog signals from the magnetic sensor 26 to digital signals for use by the computing device 14, by way of example, although the measurement device 18 may include other types and/or numbers of devices, elements, and/or components. The measurement device 18 is sized to be located on the longitudinal member 16 for advancement into the patient's body. By way of example, the measurement device 18 may be similar in size to electrodes typically employed on catheters for ablation procedures. In one example, the measurement device 18 has dimensions of approximately 1.2 mm×1.2 mm×0.5 mm, although other measurement device dimensions may be utilized that provide the ability for the measurement device 18 to be utilized within the patient's body, such as in intracardiac applications, by way of example. The measurement device 18, for example, may be a device such as the GSR sensor disclosed in Honkura, “The Development of ASIC Type GSR Sensor Driven by GHz Pulse Current,” SENSORDEVICES 2018: The Ninth International Conference on Sensor Device Technologies and Applications, (2018), the disclosure of which is incorporated by reference herein in its entirety.

In one example, the magnetic sensor 26 of the measurement device 18 is an ultrasensitive magnetic sensor configured to measure biological magnetic fields on the order of one pico Tesla, for example. The magnetic sensor 26 provides ultra-high sensitivity that is close to the sensitivity provided by SQUID devices. By way of example, the magnetic sensor 26 in one example includes a micro coil having a wire length of approximately 450 micrometers, with approximately 66 coil turns, and a thickness of 20 micrometers, although other dimensions and configurations of the coil turns may be used for the magnetic sensor 26. In this example, the magnetic sensor 26 is a three-axis magnetic sensor configured to detect magnetic flux generated from the flow of current in the area proximate to the magnetic sensor 26. Thus, the magnetic sensor 26 is configured to measure magnetic flux in three-dimensions. Since the three-axis magnetic sensor can detect direction of the flow of current, the signal can be detected regardless of the direction of the flow of the current. Thus, the magnetic sensor 26 is useful in detecting sources of abnormalities in the flow of current through the magnetic flux, such as an arrhythmia when measuring cardiac activity, by way of example only. The magnetic sensor 26 is configured to measure the magnetic flux from the flow of current in real-time.

The magnetic sensor 26 is coupled to the signal processing device 28. In this example, signal processing device 28 includes the integrated circuit 30, which is configured to serve as an analog to digital converter to convert the analog magnetic flux signals from the magnetic sensor to digital signals that provide digital representations of the magnetic flux signals for processing by the computing device 14, for example. Additionally, in some examples, the integrated circuit 30 may also include a microcontroller for performing some of the processing functions as described below, such as arranging the magnetic flux signal from the magnetic sensor 26 for display. In one example, the integrated circuit 30 is an application-specific integrated circuit (ASIC), although other types and/or numbers of signal processing devices can be employed. The integrated circuit 30 is coupled to the magnetic sensor 26 using known techniques. The integrated circuit 30 in this example is formed using MEMS technology to generate an electronic control circuit that can be miniaturized to electrode size for use with the magnetic sensor 26. This allows the measurement device 18 including the magnetic sensor 26 and the signal processing device 28 to be sized in a range that it can be employed, for example, in intracardiac measurements, while also having the required sensitivity to measure biomagnetism.

Referring again to FIGS. 1 and 2, optionally, the longitudinal member 16 in some examples may also include the positional sensor 20, which is located proximate the distal end 22 of the longitudinal member 16. In one example, the positional sensor 20 is a magnetic position sensor that is configured to measure geomagnetism, although other positional sensors that use other location techniques may be employed. For example, the positional sensor 20 may be torque or rotational sensors, or displacement sensors such as accelerometers or gyroscopes. The positional sensor 20 serves as a three-dimensional compass for determining the position of the longitudinal member 16, such as a catheter, within the patient's anatomy. The positional sensor 20 is coupled to the computing device 14, by way of example, to provide data regarding the position of the longitudinal member 16, such as a catheter. In another example, the positional sensor 20 may comprise a permanent magnet located on the longitudinal member 16 and which would be used with a magnetic sensor grid placed outside the patient's anatomy.

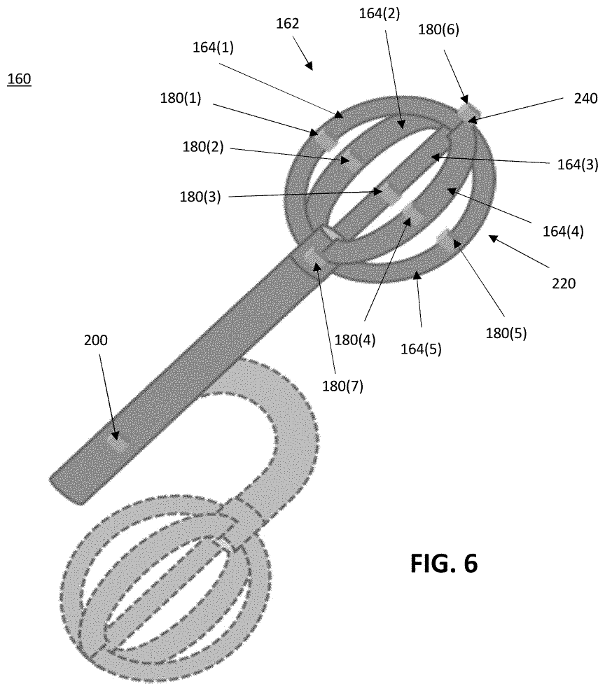

Referring now to FIG. 6, an exemplary catheter 160 that may be employed as the longitudinal member 16 in system 11 is illustrated. In this example, catheter 160 is a deflectable catheter that includes a basket-like configuration 162 on the distal end 220 having a plurality of expandable ribs 164(1)-164(5), although the basket-like configuration may have other numbers of expandable ribs. As shown in FIG. 6, the distal end 220 is deflectable between a first position and a second position. The plurality of expandable ribs 164(1)-164(5) may be delivered into the body in a compressed state and then expanded to position the basket configuration 162 within a vessel. In this example, the basket-like configuration 162 includes a plurality of measurement devices 180(1)-180(7) including magnetic sensors. The measurement devices 180(1)-180(5) are located on the expandable ribs 164(1)-164(5), respectively, while the measurement device 180(6) is located at the distal tip 240 of the catheter 160 and the measurement device 180(7) is located at the base of the basket-like configuration 162. In other examples, additional measurement devices may located in other positions. The magnetic sensors of measurement devices 180(1)-180(7) are the same in structure and operation as the magnetic sensor 26 described above. In this example, the catheter 160 also includes an additional sensor, such as position sensor 200, which is the same in structure and operation as described above with respect to position sensor 20, although other types and/or numbers of additional sensors may be employed on the catheter 160 in accordance with the present technology.

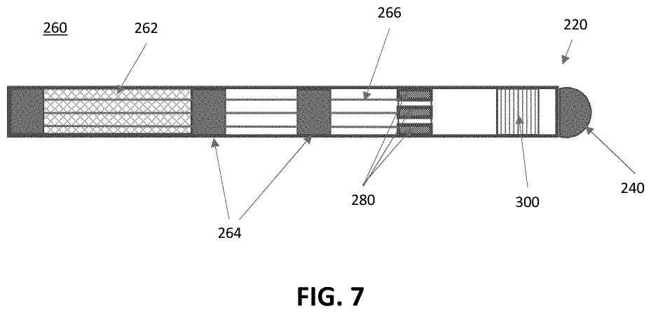

Referring now to FIG. 7, another exemplary catheter 260 that may be employed as the longitudinal member 16 in system 11 is illustrated. In this example, the catheter 260 includes a braided portion 262 near the distal end 220 that provides for greater pliability of the shaft of the catheter 260 for improved maneuverability, although the catheter 260 may have other structures and/or configurations to assist in positioning the catheter 260 in the patient's body. The catheter 260 also includes electrode rings 264, which are evenly spaced to provide evenly spaced bi-pole pairs. In this example, the catheter 260 includes a plurality of measurement devices 280, each including a magnetic sensor, located proximate to the distal end of the catheter 260. The magnetic sensor is the same in structure and operation as the magnetic sensor 26 described above. The catheter 260 also includes an additional sensor 300, that may for example be a positional sensor. The catheter 260 also includes a force contact sensor 240 that measures force applied to the distal tip. In this example, fiber optic cables 266 are used to connect to the sensors, although other techniques, such as wireless communication may be employed.

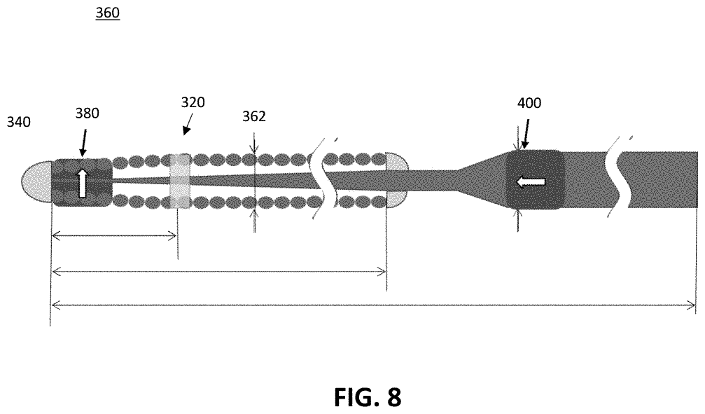

FIG. 8 is an exemplary guidewire 360 that may be employed as the longitudinal member 16 in the system 11. The guidewire 360 includes coils 362 located near the distal end 320 to assist in locating the guidewire 360 in the patient's body as well as to assist in delivering and maneuvering the guidewire. In another example, the coils 362 can additionally serve as the coils of the magnetic sensor element itself and serve as the magnetic sensor 26. The guidewire 360 includes a measurement device 380 including a magnetic sensor located near the distal tip 340 of the guidewire 360. The magnetic sensor is the same in structure and operation as the magnetic sensor 26 described above. The guidewire also includes an additional sensor, such as position sensor 400, which is the same in structure and operation as described above with respect to position sensor 20, although other types and/or numbers of additional sensors may be employed on the guidewire 360 in accordance with the present technology.

Additionally, it will be rather apparent to those skilled in the art that due to the size of the magnetic sensor 26 and/or the measurement device 18, it can readily be incorporated into any number of therapeutic devices including without limitation, PTA and PTCA balloon catheters, drug coated balloon catheters, ablation catheters, atherectomy catheters, laser catheters, ultrasound catheters, and the like to further guide or aid the therapeutic procedure. Furthermore, the magnetic sensor 26 can be incorporated into implantable devices including without limitation, stents, pacemakers, implantable cardioverter devices (ICD), and the like. In particular, in using with implantable devices, rather than a wired connection used for catheters, a wireless connection could be employed. Such wireless connection would allow the implanted devices to be monitored in real-time as well as over a period of time as necessary.

Referring now to FIGS. 1 and 4, the computing device 14 is coupled to the measurement device 18 through the integrated circuit 30 and a communication network. The computing device 14 includes at least one processor 32, a memory 34, a communication interface 35, a user input device 36, and a display interface 38, which are coupled together by a bus 39 or other link, although other types and/or numbers of systems, devices, components, parts, and/or other elements in other configurations and locations can be used.

The processor 32 of the computing device may execute programmed instructions stored in the memory for any number of the functions or other operations illustrated and described by way of the examples herein, including generating magnetic flux maps based on received magnetic flux data from the measurement device 18. The processor 32 of the computing device 14 may include one or more CPUs, or general processors with one or more processing cores, for example, although other types of processor(s) can be used.

The memory 34 of the computing device 14 stores the programmed instructions for one or more aspects of the present technology as illustrated and described herein, although some or all of the programmed instructions could be stored elsewhere. A variety of different types of memory storage devices, such as random access memory (RAM), read only memory (ROM), hard disk drive (HDD), solid state drives (SSD), flash memory, or other computer readable medium that is read from and written to by a magnetic, optical, or other reading and writing system that is coupled to the processor(s) 32 can be used for the memory 34.

Accordingly, the memory 34 of the computing device 14 can store application(s) that can include executable instructions that, when executed by the computing device 14, cause the computing device 14 to perform actions, such as to receive magnetic flux signals from the measurement device 18 and generate a mapping of the magnetic flux based on electrical activity of the heart. The application(s) can be implemented as modules or components of other application(s). Further, the application(s) can be implemented as operating system extensions, modules, plugins, or the like.

The communication interface 35 of the computing device 14 operatively couples and communicates between the computing device 14 and the integrated circuit 30 of the signal processing device 28, which are coupled together by one or more communication network(s), although other types and/or numbers of connections and/or configurations to other device and/or elements can be used. By way of example only, the communication network(s) can include local area network(s) (LAN(s)) or wide area network(s) (WAN(s)), and/or wireless networks, although other types and/or number of protocols and/or communication network(s) can be used.

The user input device 36 in the computing device 14 can be used to input selections, such as one or more parameters related to the mapping process by way of example, although the user input device 36 could be used to input other types of requests and data. The user input device 36 can include one or more keyboards, keypads, or touch screens, although other types and/or numbers of user input devices can be used.

The display interface 38 of the computing device 14 can be used to show data and information to the user. By way of example, the display interface 38 may illustrate the position of the longitudinal member 16 relative to the patient's anatomy based on a three-dimensional model generated from image data obtained from one or more imaging devices as described below. In another example, the display interface 38 may illustrate the magnetic flux measured by the measurement device 18 in real-time. The display interface 38 may be a liquid crystal display (LCD), gas plasma, light emitting diode (LED), or any other type of display interface used with a computing device. The display interface 38 may also include a touch sensitive screen arranged to receive input from an object such as a stylus or a human hand.

Although an example of the computing device 14 is described and illustrated herein, the computing device can be implemented on any suitable computer apparatus or computing device. It is to be understood that the apparatuses and devices of the examples described herein are for exemplary purposes, as many variations of the specific hardware and software used to implement the examples are possible, as will be appreciated by those skilled in the relevant art(s).

Furthermore, each of the devices of the examples may be conveniently implemented using one or more general purpose computers, microprocessors, digital signal processors, and micro-controllers, programmed according to the teachings of the examples, as described and illustrated herein, and as will be appreciated by those of ordinary skill in the art.

The examples may also be embodied as one or more non-transitory computer readable media having instructions stored thereon for one or more aspects of the present technology as described and illustrated by way of the examples herein, which when executed by a processor, cause the processor to carry out the steps necessary to implement the methods of the examples, as described and illustrated herein.

Referring again to FIG. 1, the computing device 14 is coupled to and configured to receive data from one or more imaging devices 40 such as a CT scanner, x-ray machine, or an MRI device, by way of example only. For example, the computing device 14 is coupled to the one or more imaging devices 40 by one or more communication networks. The computing device 14 may receive data from the one or more imaging devices 40, although the computing device may alternatively receive the data from other sources, such as other server devices coupled to the one or more imaging devices 40. The data may include image data, such as CT, MRI, or x-ray image data, related to the portion of the patient's anatomy for which the mapping described below is to be performed. By way of example, the image data may be related to the patient's heart for performing cardiac activity mapping, although image data for other tissues or organs may be utilized.

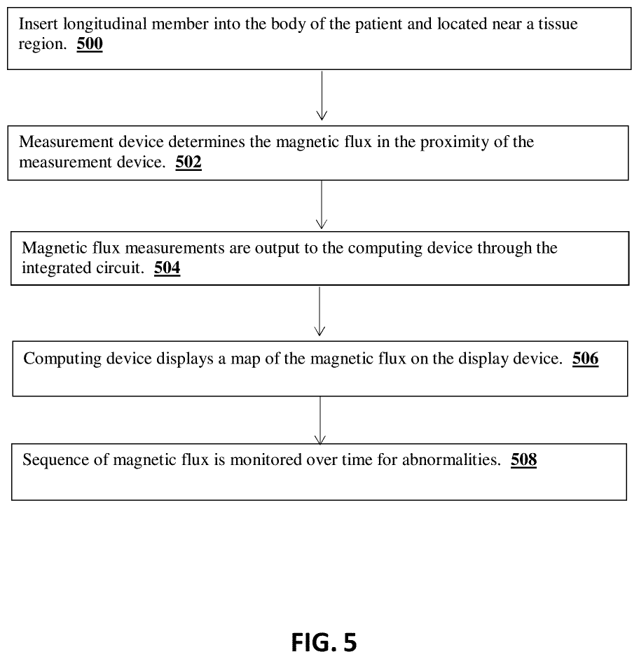

An exemplary method for cardiac mapping using the system of the present technology will now be described with reference to FIGS. 1-5. It is to be understood that the longitudinal member 16 could be any of the exemplary catheters shown in FIGS. 6-8. Although cardiac mapping is described, it is to be understood that the system of the present technology could be employed to map the electrical activity of other portions of a patient's anatomy, such as other tissues or organs. Referring more specifically to FIG. 5, in step 500, the longitudinal member 16 is inserted into the body of the patient and located near a tissue region. The tissue region may be any portion of a tissue of the patient such as by way of example only, various organs, body lumens or cavities, such as various ducts or vessels, or blood vessels. In one example, the distal end 22 of the longitudinal member 16 is located near the endocardial membrane of the patient's heart, although the distal end 22 of the longitudinal member 16 may be located in other intracardiac locations. The longitudinal member 16 may be placed relative to and near the tissue region using various approaches and orientations. In this example, the positional sensor 20 is used to determine the three-dimensional positioning of the longitudinal member 16 based on the earth's magnetic field or an externally generated magnetic field, as well as a three-dimensional model of the patient's anatomy generated from image data from the one or more imaging devices 40, although other positioning techniques may be employed.

Next, in step 502, the magnetic sensor 26 of the measurement device 18 determines the magnetic flux in the proximity of the measurement device 18. In other examples, additional magnetic sensors may be employed. For example, the magnetic sensor 26 of the measurement device 18 may obtain the magnetic flux resulting from cardiac activity. In one example, the measurement device 18 measures the generated magnetic field from the patient's heart during cardiac excitation. The magnetic sensor 26 of the measurement device 18 is configured to measure the magnetic flux in three-dimensions. The magnetic sensor 26 is also configured to measure changes in the magnetic flux in real-time.

In step 504, the magnetic flux measurements are output to the computing device through signal processing device 28. In one example, the signal processing device 28 includes the integrated circuit 30, which is configured to serve as an analog to digital converter to convert the analog magnetic signals to digital signals for processing by the computing device 14, for example, although the conversion may take place in other locations, and the signal processing device 28 may include other integrated circuits configured for providing other processing of the magnetic flux signals, such as amplification or filtering, by way of example only. In one example, the signal processing device 28 may also include a microcontroller that does some processing of the digital representations of the magnetic flux signals.

Next, in step 506, the computing device 14 displays a map of the magnetic flux on the display interface 38. The computing device 14 determines the directionality and intensity of the magnetic flux to provide the mapping of the magnetic distribution. By way of example, the magnetic distribution may be displayed in three dimensions. In one example, the magnetic flux from the measurement device 18 could be combined with data from the one or more imaging devices 40, such as an ECG, for displaying the magnetic flux over the results from the ECG. This allows for simultaneously displaying the magnetic flux distribution on the heart cross-section, when utilized to map cardiac activity. The magnetic distribution may be correlated to the electrical activity of the tissue being monitored, such as the heart.

In step 508, the sequence of magnetic flux is monitored by the computing device 14 over time for abnormalities, such as an arrhythmia by way of example only. The changes in the magnetic flux are monitored in real-time. The three-dimensional magnetic flux data may be utilized to determine the location of the arrhythmia. The source of arrhythmia could be diagnosed from the sequence and tachycardia of the abnormality in the magnetic flux distribution. The location data for the abnormality may then be utilized for treatment of the abnormality, such as by ablation using a separate catheter device.

Accordingly, as illustrated and described above by way of the examples herein, this technology provides an intracardiac catheter device and methods of use thereof for mapping cardiac activity using magnetophysiology.

Having thus described the basic concept of the invention, it will be rather apparent to those skilled in the art that the foregoing detailed disclosure is intended to be presented by way of example only and is not limiting. Various alterations, improvements, and modifications will occur and are intended to those skilled in the art, though not expressly stated herein. These alterations, improvements, and modifications are intended to be suggested hereby, and are within the spirit and scope of the invention. Additionally, the recited order of processing elements or sequences, or the use of numbers, letters, or other designations therefore, is not intended to limit the claimed processes to any order except as may be specified in the claims. Accordingly, the invention is limited only by the following claims and equivalents thereto.

What is claimed is:

1. An apparatus comprising:
a longitudinal member having a proximal end and a distal end, the longitudinal member configured to be located near a tissue region in a body of a patient;
a measuring device configured and sized to be located proximal to the distal end of the longitudinal member, the measuring device comprising:
a magnetic sensor configured to measure biomagnetism and output magnetic flux data; and
a signal processing device coupled to the magnetic sensor and configured to convert the output magnetic flux data to a digital representation of the output magnetic flux data.

2. The appartus of claim 1 further comprising:
a computing device communicatively coupled to the signal processing device to receive the digital magnetic flux data, the computing device comprising a processor coupled to a memory and configured to execute programmed instructions stored in the memory to:
receive, from the measuring device, magnetic flux data based on electrical activity near the tissue region; and
generate a magnetic flux distribution for the tissue region based on the magnetic flux data.

3. The apparatus of claim 2, wherein the processor is further configured to execute at least one additional programmed instruction stored in the memory to:
generate a magnetix flux distribution map based on the magnetic flux distribution for the tissue region;
display the magnetic flux distribution map for the tissue region in a three-dimensional representation.

4. The apparatus of claim 2, wherein the received magnetic flux data is three-dimensional.

5. The apparatus of claim 2, wherein the received magnetic flux data is received in real-time.

6. The apparatus of claim 5, wherein the magnetic flux distribution is generated in real-time.

7. The apparatus of claim 1, wherein wherein the longitudinal member is a catheter or a micro catheter, or a guidewire.

8. The apparatus of claim 1, wherein the magnetic sensor is configured to measure magnetic signals on the order of one nano Tesla (nT).

9. The apparatus of claim 1, wherein the magnetic sensor is configured to measure magnetic signals on the order of one pico Tesla (pT).

10. The apparatus of claim 1, wherein the longtidunal member further comprises a positional sensor located proximate to the distal end configured to measure the position of longitudinal member within the patient's anatomy.

11. The apparatus of claim 10, wherein the positional sensor is a magnetic sensor configured to measure geomagnetism.

12. The apparatus of claim 10, wherein the processor is configured to execute at least one additional programmed instruction stored in the memory to:
receive, from the positional sensor, location data for the longitudinal member; and
display the location of the longitudinal member on a three-dimensional model of a least a portion of the tissue region.

13. The apparatus of claim 1, wherein the measurement device is encapsulated in a distal tip of the longtidunal member.

14. The apparatus of claim 1, wherein the longitudinal member further comprises a permanent magnet located proximate to the distal end and a positional sensor comprising a magnetic sensor grid located outside the patient's anatomy.

15. A method for measuring electrical activity, the method comprising:
receiving, by a computing device, magnetic flux data from a measuring device positioned on a longitudinal member having a proximal end and a distal end, wherein the longitudinal member is configured to be located near a tissue region in a body of a patient and the measuring device is located proximate to the distal end, wherein the magnetic flux data is based on electrical activity near the tissue region; and
generating, by the computing device, a magnetic flux distribution for the tissue region based on the magnetic flux data.

16. The method of claim 15 further comprising:
generating a magnetix flux distribution map based on the magnetic flux distribution for the tissue region;
displaying the magnetic flux distribution map for the tissue region in a three-dimensional representation.

17. The method of claim 15, wherein the received magnetic flux data is three-dimensional.

18. The method of claim 15, wherein the received magnetic flux data is received in real-time.

19. The method of claim 18, wherein the magnetic flux distribution is generated in real-time.

20. The method of claim 15, wherein the magnetic sensor is configured to meaure magnetic signals on the order of one nano Tesla (nT).

21. The method of claim 15, wherein the magnetic sensor is configured to measure magnetic signals on the order of one pico Tesla (pT).

22. The method of claim 15 further comprising:
receiving, by the computing device, location data for the longitudinal member from a positional sensor located proximate to the distal end, wherein the positional sensor is a magnet configured to measure geomagnetism; and
displaying the location of the longitudinal member on a three-dimensional model of a least a portion of the tissue region.

23. The method of claim 15, wherein the tissue region is a portion of the patient's heart.

24. The method of claim 15, wherein the measurement device is encapsulated in a distal tip of the longtidunal member.
